SEQUENCE LISTING (13 sequences)

SEQ ID NO: 1 - DNA, 4153 bp, Artificial Sequence
  Solanum lycopersicum
gtagatgttcttgtcatgatgacttccaggttttgggaataatagatgttgaatattgatagttattgaattggtttttattaatgagtttaagtcttccgcattactttttgttgttattacattgaaatgttaaggtttagattggttggttcgctcacataggagggtaagtgtgggtgccagtcgcggcccggatttgggtcgtgacataaaggttgggactggttcatgtaaattctgttggttgtttgacgctactattttaagtatttctatacttagtcaatttttgtgggtgcaagtaggctgagtgttacagacgtttgtaagactgcctagagtggtgtgcgataaaaataatcgaccctctttcagatagcgttgcagtatgtgttgcttctttttttttttgcagggacaatccgtagttgttgactccgtcattttttaaactcggtttagtagtgaaaactgaaaactcagtttaggtaacatgagatcctttatctaaaattcccagtttctaactttgctttcatcttcaaatgataaatagagtgttagattcatgaagtaatcatggattcaagttagaatcacttaactcaatcttgtgggtgaaaatcaagaacagaatgtcaaatgaaattactcaaatgaagggctggtttgttcaacttgaaccacgggtgaattgaaatagccatctatcattttctttcagtactctcgttatgatttccagctacgtcccatgctatgtattaaaatgattagtagttctatgacttaggcctcatttgtttacacttgatgaagtttgaaactcaatcattcacacattgggctattaagtgcattttaaaaaaaaatcaaatctgaaagcatctctatgagagagtcttatcatgtttaccatggatgtttctcttaattggtagtggttcgagggatgccatgtcacaacgttaagtttcagatttgggttgtggaatatgttgaaaccaagttcctatttcggccttggtttgtttagttggttcagcgcaatctgtttagtttgatcctacatgagtagacttggtccaacagcccatatttatgtgaattttcagattccttttattgtactgcatttgtgtctaaatataaatagtatatactatagtcaattttacgagctgacgttacctattcctcttctctcatttaaaacaagtaatcttatttacttgatgagtgtcgtagtttgaatgatgatctaactcatagaacaaatggttgctgctcactatcatattggaattgaccaaaaaaataaatgaatgcaccttctgatttcattattatcaataacaagtaggaatgcagaaaacatgactaacaaatggacaacttacttctaacaacaagtggatcacaaacattgtgtgtcttccttcatcctataacattgtgaaatcttacatttccagtttaaacagcttaaatttcagcatccatattccccaccttcctgccaccgatgcctatcaacgatcctcacatggagaagaggatccaattgataagaagccagcttgtttcacaacgtcctcacttgtttcttctttccctagcctttgctttccttgtggcagatccatttggtttaagtccagtaggggacactgactttaatccagtaaagaatgacattgcaccatacaaacaagtcatggaaagttggcatggagacaagaataatcggttaggcctcggcaatctggagtttgttaatgaaatttacggtcctgaatcattagaatttgatattttgggtcatggcccatatgccggactagctgatgggcgtattgtacggtggatgggggaggatgctggttgggaaacatttgcagtcgtcacacgtaactggtaagcatctgaattatatacctttatggacatattgaatatgttgtgaagatggatataattgcaatgtatttttgtttaggaagtgggaggatagagtgtatatatatatactctatagtattattcttttaaatttcatttatctccctgtcgttgtcgtataaggtcggagaagctttgtgctaaaggaaaggattcaacaacacctaagcaatatagagttgagccagaatgtggcaggccccttgggctaaggttcaataaacagacaggggatttgtacatagcagatgcatactatggactcctagttgtgggtcctgaaggaggcgtggcaacgtccttggctacacatgttgaagggaaacgaatactcttcgccaacgaccttgatatccatacaaatggctccatcttcttcacagacactagcaagaaatacaacagagtgtaggttaacaaatcttttttttatcaaatactacaattgtagtggtttatattactcttctttcattatataaatttcttatgtaggaaccattttctcataatgttagaaggagaagacagtggtagacttcttaggtatgatattgctacgaaatctactaatgttgtcttggatggattgacgttccctaacggagtacaattatccaaggatcaaacttttcttctcttcactgaaacaaccaattgcaggtatccatcatccctctacaagtgtttgtttaattcagacgtaaatcacctaaacgtgtaattcaccgagacaacaaatattataaataccttgctgttatatgttcaggctgatgaaatactggatagaaggtccaaagagagggatagttgagattgttgcaaaccttccaggatttccagacaacgtaagggtgaacgaaaaaggtcaattctgggttgcaatagattgttgtcgaactcgtgcacaagaagtgctgataaataatccatggatgagaagcatttactttagattaccgattcagatgcgttacctggctagattgatggggatgaaaatgtacactgttatatcactcttcaatgaaaatggagaaattattgatgttcttcaagataaaaaaggtgtagttatgaagttggttagtgaagttagagaagtaaatggcaagctatggattgggactgtggctcataaccatattgctacccttccatacccataaataaaactacgttttgccttcccttttttttataaattaattttgcaatcttagtctggcttccttcatttatgtgtaacaaaactaatgaataataccccaacacttgtttaacccattcttcacatatttttttatactagtcttagaaacgtgcgttgcaagtttgatccctactattatataaaacttatataagcctaacttcatagatataaaaatgatcgcgtttactaattaagctgttaacatcccaaaagattgattgcagtctccttgaagataccaacatgccttctctcattgcaacctccattagaagtagtaagaatttgtacactatatattttgtttctgtaagcaacaaaagaatatatgagtaataaagtgcttatttttataagaaaaaagctaaacctaataatagtgaaagataaaaccaacagaaatatagtgacagttagagatggaactttttttgtttcttccaagtgggaaaaagtctcttcataatgtatacaatgacaccatttataggatatgattaagtaaaatcaaagagtgttttaaatatgacattaatttcttgagaatgtggaaaagtttatacaagtgtgaaagattaatgaggagatagtcaaggtgtatagtattaatacacataacgaaaaaaaattgacctagaaattattgagttgcatttttaaacaatatatttacaataacgttgtagtattttatttttttatattttgcatctgatggagaaatgaaaacttttttaagagcctttactaatattataattaattgtttttacaatacttcatttttgtattttattatatcaaaataagtgaattataaatatcagatttctgtaaatgaagaaagtgtaatgggatgataaagtgttaaagtatatatttagtgaaagctttattaagaagacaaacatgcaaatattaatctaaaaattcaattctatattgagttttgaaatgcatatcaatgac

SEQ ID NO: 2 - DNA, 19 bp, Artificial Sequence
  Synthesized
gggaaagaagaaacaagtg

SEQ ID NO: 3 - DNA, 23 bp, Artificial Sequence
  Synthesized
attggggaaagaagaaacaagtg

SEQ ID NO: 4 - DNA, 23 bp, Artificial Sequence
  Synthesized
aaaccacttgtttcttctttccc

SEQ ID NO: 5 - DNA, 20 bp, Artificial Sequence
  Synthesized
tcaactgagcaaagacacct

SEQ ID NO: 6 - DNA, 20 bp, Artificial Sequence
  Synthesized
ctcgtacagcagagagtgtt

SEQ ID NO: 7 - DNA, 22 bp, Artificial Sequence
  Synthesized
tctccgaccagttacgtgtgac

SEQ ID NO: 8 - DNA, 23 bp, Artificial Sequence
  Synthesized
atgcctatcaacgatcctcacat

SEQ ID NO: 9 - DNA, 4154 bp, Artificial Sequence
  Solanum lycopersicum
gtagatgttcttgtcatgatgacttccaggttttgggaataatagatgttgaatattgatagttattgaattggtttttattaatgagtttaagtcttccgcattactttttgttgttattacattgaaatgttaaggtttagattggttggttcgctcacataggagggtaagtgtgggtgccagtcgcggcccggatttgggtcgtgacataaaggttgggactggttcatgtaaattctgttggttgtttgacgctactattttaagtatttctatacttagtcaatttttgtgggtgcaagtaggctgagtgttacagacgtttgtaagactgcctagagtggtgtgcgataaaaataatcgaccctctttcagatagcgttgcagtatgtgttgcttctttttttttttgcagggacaatccgtagttgttgactccgtcattttttaaactcggtttagtagtgaaaactgaaaactcagtttaggtaacatgagatcctttatctaaaattcccagtttctaactttgctttcatcttcaaatgataaatagagtgttagattcatgaagtaatcatggattcaagttagaatcacttaactcaatcttgtgggtgaaaatcaagaacagaatgtcaaatgaaattactcaaatgaagggctggtttgttcaacttgaaccacgggtgaattgaaatagccatctatcattttctttcagtactctcgttatgatttccagctacgtcccatgctatgtattaaaatgattagtagttctatgacttaggcctcatttgtttacacttgatgaagtttgaaactcaatcattcacacattgggctattaagtgcattttaaaaaaaaatcaaatctgaaagcatctctatgagagagtcttatcatgtttaccatggatgtttctcttaattggtagtggttcgagggatgccatgtcacaacgttaagtttcagatttgggttgtggaatatgttgaaaccaagttcctatttcggccttggtttgtttagttggttcagcgcaatctgtttagtttgatcctacatgagtagacttggtccaacagcccatatttatgtgaattttcagattccttttattgtactgcatttgtgtctaaatataaatagtatatactatagtcaattttacgagctgacgttacctattcctcttctctcatttaaaacaagtaatcttatttacttgatgagtgtcgtagtttgaatgatgatctaactcatagaacaaatggttgctgctcactatcatattggaattgaccaaaaaaataaatgaatgcaccttctgatttcattattatcaataacaagtaggaatgcagaaaacatgactaacaaatggacaacttacttctaacaacaagtggatcacaaacattgtgtgtcttccttcatcctataacattgtgaaatcttacatttccagtttaaacagcttaaatttcagcatccatattccccaccttcctgccaccgatgcctatcaacgatcctcacatggagaagaggatccaattgataagaagccagcttgtttcacaacgtcctcactttgtttcttctttccctagcctttgctttccttgtggcagatccatttggtttaagtccagtaggggacactgactttaatccagtaaagaatgacattgcaccatacaaacaagtcatggaaagttggcatggagacaagaataatcggttaggcctcggcaatctggagtttgttaatgaaatttacggtcctgaatcattagaatttgatattttgggtcatggcccatatgccggactagctgatgggcgtattgtacggtggatgggggaggatgctggttgggaaacatttgcagtcgtcacacgtaactggtaagcatctgaattatatacctttatggacatattgaatatgttgtgaagatggatataattgcaatgtatttttgtttaggaagtgggaggatagagtgtatatatatatactctatagtattattcttttaaatttcatttatctccctgtcgttgtcgtataaggtcggagaagctttgtgctaaaggaaaggattcaacaacacctaagcaatatagagttgagccagaatgtggcaggccccttgggctaaggttcaataaacagacaggggatttgtacatagcagatgcatactatggactcctagttgtgggtcctgaaggaggcgtggcaacgtccttggctacacatgttgaagggaaacgaatactcttcgccaacgaccttgatatccatacaaatggctccatcttcttcacagacactagcaagaaatacaacagagtgtaggttaacaaatcttttttttatcaaatactacaattgtagtggtttatattactcttctttcattatataaatttcttatgtaggaaccattttctcataatgttagaaggagaagacagtggtagacttcttaggtatgatattgctacgaaatctactaatgttgtcttggatggattgacgttccctaacggagtacaattatccaaggatcaaacttttcttctcttcactgaaacaaccaattgcaggtatccatcatccctctacaagtgtttgtttaattcagacgtaaatcacctaaacgtgtaattcaccgagacaacaaatattataaataccttgctgttatatgttcaggctgatgaaatactggatagaaggtccaaagagagggatagttgagattgttgcaaaccttccaggatttccagacaacgtaagggtgaacgaaaaaggtcaattctgggttgcaatagattgttgtcgaactcgtgcacaagaagtgctgataaataatccatggatgagaagcatttactttagattaccgattcagatgcgttacctggctagattgatggggatgaaaatgtacactgttatatcactcttcaatgaaaatggagaaattattgatgttcttcaagataaaaaaggtgtagttatgaagttggttagtgaagttagagaagtaaatggcaagctatggattgggactgtggctcataaccatattgctacccttccatacccataaataaaactacgttttgccttcccttttttttataaattaattttgcaatcttagtctggcttccttcatttatgtgtaacaaaactaatgaataataccccaacacttgtttaacccattcttcacatatttttttatactagtcttagaaacgtgcgttgcaagtttgatccctactattatataaaacttatataagcctaacttcatagatataaaaatgatcgcgtttactaattaagctgttaacatcccaaaagattgattgcagtctccttgaagataccaacatgccttctctcattgcaacctccattagaagtagtaagaatttgtacactatatattttgtttctgtaagcaacaaaagaatatatgagtaataaagtgcttatttttataagaaaaaagctaaacctaataatagtgaaagataaaaccaacagaaatatagtgacagttagagatggaactttttttgtttcttccaagtgggaaaaagtctcttcataatgtatacaatgacaccatttataggatatgattaagtaaaatcaaagagtgttttaaatatgacattaatttcttgagaatgtggaaaagtttatacaagtgtgaaagattaatgaggagatagtcaaggtgtatagtattaatacacataacgaaaaaaaattgacctagaaattattgagttgcatttttaaacaatatatttacaataacgttgtagtattttatttttttatattttgcatctgatggagaaatgaaaacttttttaagagcctttactaatattataattaattgtttttacaatacttcatttttgtattttattatatcaaaataagtgaattataaatatcagatttctgtaaatgaagaaagtgtaatgggatgataaagtgttaaagtatatatttagtgaaagctttattaagaagacaaacatgcaaatattaatctaaaaattcaattctatattgagttttgaaatgcatatcaatgac

SEQ ID NO: 10 - DNA, 46 bp, Artificial Sequence
  Synthesized
gaaggtgaccaagttcatgctaaaggctagggaaagaagaaacaag

SEQ ID NO: 11 - DNA, 47 bp, Artificial Sequence
  Synthesized
gaaggtcggagtcaacggattcaaaggctagggaaagaagaaacaaa

SEQ ID NO: 12 - DNA, 28 bp, Artificial Sequence
  Synthesized
gatccaattgataagaagccagcttgtt

SEQ ID NO: 13 - protein, 414 aa, Artificial Sequence
  Solanum lycopersicum
MPINDPHMEKRIQLIRSQLVSQRPHLFLLSLAFAFLVADPFGLSPVGDTDFNPVKNDIAPYKQVMESWHGDKNNRLGLGNLEFVNEIYGPESLEFDILGHGPYAGLADGRIVRWMGEDAGWETFAVVTRNWSEKLCAKGKDSTTPKQYRVEPECGRPLGLRFNKQTGDLYIADAYYGLLVVGPEGGVATSLATHVEGKRILFANDLDIHTNGSIFFTDTSKKYNRVNHFLIMLEGEDSGRLLRYDIATKSTNVVLDGLTFPNGVQLSKDQTFLLFTETTNCRLMKYWIEGPKRGIVEIVANLPGFPDNVRVNEKGQFWVAIDCCRTRAQEVLINNPWMRSIYFRLPIQMRYLARLMGMKMYTVISLFNENGEIIDVLQDKKGVVMKLVSEVREVNGKLWIGTVAHNHIATLPYP

(19) United States
(12) Patent Application Publication
(10) Pub. No.: US 2021/0095308 A1
(43) Pub. Date: Apr. 1, 2021

(54) A METHOD FOR CREATING MALE STERILE LINE OF TOMATO THROUGH GENOME EDITING AND APPLICATION THEREOF

(71) Applicants: BEIJING ACADEMY OF AGRICULTURE AND FORESTRY SCIENCES, Beijing (CN); Institute of Genetics and Developmental Biology, Chinese Academy of Sciences, Beijing (CN)

(72) Inventors: Changbao LI, Beijing (CN); Minmin DU, Beijing (CN); Chuanyou LI, Beijing (CN); Lei DENG, Beijing (CN); Ming ZHOU, Beijing (CN); Changlong WEN, Beijing (CN)

(21) Appl. No.: 16/623,659

(22) PCT Filed: Nov. 20, 2017
(86) PCT No.: PCT/CN2017/111859

§ 371 Date: Dec. 17, 2019

(30) Foreign Application Priority Data
Jun. 29, 2017 (CN) 201710514085.9

(51) Int. Cl. C12N 15/82; C12Q 1/686; C12Q 1/6895

(52) U.S. Cl. CPC C12N 15/8289; C12N 15/8213; C12Q 2600/156; C12Q 1/6895; C12Q 2600/13; C12Q 1/686

(57) ABSTRACT

The present invention uses the CRISPR/Cas9 genome editing technology to rapidly create a male sterile line of tomato and application thereof, The present invention uses the CRISPR/Cas9 genome editing technology to edit the Solyc03g053130 gene of tomato, and then obtains a male sterile mutant which is homozygous and does not contain the CAS9 transgene by self-crossing. The present invention also discloses a method for assisting in identification of a male sterile plant, which is to detect the genotype of SNP1606 in the Solyc03g053130 gene in the genome of tomato, and if the genotype of SNP1606 of the genome of the tomato to be tested is homozygous T/T, the tomato to be tested is a male sterile plant or is a candidate male sterile plant. The male sterile line of tomato and the method for detecting male sterile plants created by the present invention can be applied to other tomato strains, and have great application prospect and economic value in breeding.

### RELATED APPLICATIONS

The present application is a National Phase of International Application Number PCT/CN2017/111859, filed Nov. 20, 2017, and claims the priority of China Application No. 201710514085.9, filed Jun. 29, 2017.

### INCORPORATION BY REFERENCE

The sequence listing provided in the file entitled C6351-013_SEQUENCE_LISTING_v2_2020-07-21.txt, which is an ASCII text file that was created on Jul. 21, 2020, and which comprises 16,623 bytes, is hereby incorporated by reference in its entirety.

### TECHNICAL FIELD

The present invention belongs to the field of biotechnology, and particularly relates to a method for creating a male sterile line of tomato through genome editing and an application thereof.

### BACKGROUND ART

Tomato (Solanum lycopersicum), belonging to the family Solanaceae and the genus Lycopersicon, is an important vegetable crop widely cultivated in the world wide. It is also a favorite fruit substitute in people's daily life and plays an important role in the vegetable production in green house in China. As a strict self-pollination crop, tomato has obvious heterosis, and the hybrid has high uniformity and strong resistance. Therefore, hybrids are mainly used in production. At present, the hybrid seed production of tomato is mainly carried out by manual emasculation and pollination. This method has higher labor cost and is prone to produce impure seed and other seed safety problems. The introduction of male sterile lines during seed production can optimize seed production procedures, reduce labor costs, increase hybrid seed purity and avoid losing parents. Therefore, the breeding of male sterile lines of tomato is of great significance. Male sterility refers to a phenomenon in which the female organs of plants are normal and the male organs are abnormal due to physiological or genetic reasons during the sexual reproduction, and no pollen can be produced or pollen abortion occurs and therefore pollination is impossible. Male sterility is mainly divided into two types: cytoplasmic sterility and nuclear sterility (Wang Chao et al., 2013; Yang Lifang et al., 2013; Ma Xiqing et al., 2013). As early as the 1930s, people began research on male sterility in tomatoes. To date, more than 60 male sterile materials of tomato have been reported worldwide, all of which belong to the “nuclear sterility type” controlled by nuclear genes (Susan et al., 1997; Chen Yuhui et al., 2004; Xing Hucheng et al.. 2004). These include at least 3 materials belonging to the functional sterility type (anther dehiscence is poor or stigma is exposed), 6 materials belonging to the structural sterility type (stamens are degenerated or absent) and more than 40 materials belonging to the pollen abortion type (pollen development is defective). No cytoplasmic sterility type of male sterile material of tomato was found in natural resources, but some cytoplasmic male sterility types were obtained by distant hybridization or genetic engineering and other means. The various types of male sterile materials of tomato currently available in natural resources have their own advantages and disadvantages, which limits their application in hybrid seed production. (1) Although the stigma exposure type is difficult to self-pollinate, in the case of group planting, pollination between adjacent plants and adjacent flowers is easy to occur. From the perspective of sterile line application and seed production, this sterility type cannot avoid the possibility of self-crossing and is prone to seed problems; and this type of sterility is susceptible to environmental influences, resulting in poor stability of sterility. (2) The anther indehiscence type can basically avoid the possibility of self-crossing, but because its style is shorter than the anther tube of the stamen, it cannot be directly pollinated and still needs to be emasculated. The operation is complicated, and some combinations show the disadvantages of low combining ability and late maturation, and such sterile lines are only applied in Bulgaria, Czech Republic and other eastern European countries. (3) No stamen or stamen degeneration type can also completely avoid self-crossing, but the setting percentage of such material is low, the seed content in the fruit is small, and the agronomic traits are generally poor and thus it is difficult to be applied (Susan et al., 1997; Chen Yuhui et al., 2004; Xing Hucheng et al., 2004). (4) More than 40 materials belonging to the pollen abortion type found in natural resources have great potential for application in tomato hybrid seed production. These materials are generally controlled by a pair of recessive nuclear genes and closely linked molecular markers are needed to assist in the identification of sterile traits and trans-breeding. Since most of the sterile genes have not vet been cloned, the application of such sterile materials in hybrid seed production is greatly limited.

Another factor that restricts the widespread application of recessive nuclear sterile lines in tomato hybrid seed production is that it is difficult to find an effective maintainer line, and it is impossible to produce a large number of sterile line seeds for seed production, and these lines can only be preserved in the form of hybrids. The sterile plant as female parent was hybridized with the heterozygous fertile plant as male parent. Their offspring were separated into homozygous sterile plants and heterozygous fertile plants in a ratio of 1:1. This method can be used to multiply a mixed population of sterile plants and fertile plants. This population has both sterile plants and heterozygous plants that maintain infertility, so it is called a dual-use system. How to quickly and accurately select male sterile plants from the dual-use system as a female parent for seed production has become a technical problem to be solved urgently. The localization and cloning of male sterile genes can be carried out, and the selection of sterile plants can be carried out efficiently and accurately by using molecular markers, but this method increases the cost and technical difficulty, and only three male sterile mutants ps-2, ms-10 and ms-15 are currently applicable to this method. In addition, people also tried to introduce some seedling marker traits closely linked to male sterility into the sterile line, and use the seedling marker traits to assist in the selection of sterile plants. The male sterile mutant ms-10 which is widely used at present and the green stem an are closely linked, and the selection efficiency of the male sterile mutant by the seedling marker trait an can reach 90% (Jeong et al., 2014; Zhang, L. et al. 2016). The disadvantage of this method is that it cannot guarantee 100% accuracy, and needs to use a secondary selection or recessive seedling marker traits to identify false hybrids in hybrids at a later stage.

Gene editing technology is a genetic manipulation technology that can modify DNA sequences at the genomic level. The principle of this technology is as follows: an artificial endonuclease is constructed and the artificial endonuclease cleaves DNA at a predetermined genomic site, and the cleaved DNA is mutated during the repair process by the DNA repair system, thereby achieving the purpose of site-directed genome modification. Clustered Regularly Interspaced Short Palindromic Repeats/Cas (CRISPR/Cas) is an adaptive immune defense formed by bacteria and archaea during long-term evolution and can be used against invading viruses and foreign DNA. In recent years, the type II CRISPR/Cas system has been transformed into a third generation gene editing technology, namely CRISPR/Cas9 technology (Hsu et al., 2014; Lander, 2016). In theory, CRISPR/Cas9 technology can operate on any gene of any species, and achieve rapid and accurate improvement of the target traits of the core parents without the linkage drag problem which is common in traditional backcross breeding and other problems, and this technology has shown great application prospects in crop genetics and breeding. (Huang et al., 2016).

### SUMMARY OF THE INVENTION

It is an object of the present invention to provide a method for breeding a male sterile line of tomato.

The method for breeding a male sterile line of tomato provided by the present invention comprises the following steps: using a CRISPR/Cas9 system to edit a fertility gene in the genome of a recipient tomato, thereby losing the function of the fertility gene and obtaining the male sterile line of tomato.

In the above method, the fertility gene is a gene encoding a Solyc03g053130 protein; the Solyc03g053130 protein is the following 1) or 2):

1) a protein with the amino acid sequence set forth in SEQ ID NO: 13;

2) a protein derived from 1), which is obtained by substitution, deletion and/or addition of one or more amino acid residues in the amino acid sequence set forth in SEQ ID NO: 13.

In the above methods, the CRISPR/Cas9 system comprises a sgRNA; the target sequence of the sgRNA is the DNA molecule set forth in SEQ ID NO: 2.

In the above methods, the editing method is introducing a vector for tomato genome editing into the recipient tomato. The vector for tomato genome editing contains the encoding gene of the sgRNA and the encoding gene of the Cas9 protein. In a specific embodiment of the present invention, the vector for tomato genome editing is pKSE401-sgRNA., which is a vector obtained by inserting the DNA molecule set forth in SEQ ID NO: 2 between the Bsa I restriction sites of pKSE401 vector and keeping the other sequences of pKSE401 vector unchanged.

The method for breeding a male sterile line of tomato of the present invention further comprises the step of screening a homozygous Solyc03g053130 mutant. Since tomato is a diploid plant, when Cas9 acts to start cutting the specific Solyc03g053130 gene, both alleles on two homologous chromosomes in the same cell may be edited. Homozygous SolycO3g053130 mutant refers to a plant in which the two Solyc03g053130 genes of two homologous chromosomes have the same mutation and do not carry any foreign DNA fragments. The screening method is specifically as follows: PCR amplification and sequencing of a T0 generation regenerated tomato plant are conducted using the primer pair set forth in SEQ ID NO: 7 and SEQ ID NO: 8; the T0 generation regenerated tomato plant having a nucleotide deletion or insertion in the target sequence compared to the wild-type plant is selected, and this plant is a plant with an edited Solyc03g053130 gene; the plant with an edited Solyc03g053130 gene is self-crossed, and the seeds are harvested, and the obtained seeds are sown; after the euphylla comes out, the Cas9 gene fragment is subjected to PCR cloning and electrophoresis using the primer pair set forth in SEQ NO: 5 and SEQ ID NO: 6, the T1 generation regenerated tomato plant not carrying the Cas9 gene fragment is selected, and the Solyc03g053130 gene is subjected to PCR amplification and electrophoresis using the primer pair set forth in SEQ ID NO: 7 and SEQ ID NO: 8, the plant with a homozygous mutation in the target sequence (the two Solyc03g053130 genes of two homologous chromosomes have the same mutation) and without Cas9 gene fragment is selected, and this plant is the homozygous Solyc03g053130 mutant.

In the above methods, the recipient tomato is the wild-type tomato Moneymaker. The male sterile line of tomato obtained by the above methods is a homozygous Solyc03g053130 mutant plant (individual plant No. T₀-3-6), which is obtained by inserting a thymine (T) between position 1605 and position 1606 of each Solyc03g053130 gene of the two homologous chromosomes of the wild-type tomato Moneymaker and keeping the other sequences of the genome of the wild-type tomato Moneymaker unchanged. Since the target sequence is the reverse complementary sequence of positions 1601-1619 of SEQ ID NO: 1, it results in the insertion of a thymine (T) between position 1605 and position 1606 in the corresponding Solyc03g053130 gene sequence and a frameshift mutation in the first exon, and the mutant sequence is set forth in SEQ ID NO: 9 of the sequence listing.

Another object of the present invention is to provide a biological material of any one of the following (1) to (4):

(1) the above vector for tomato genome editing;

(2) a microorganism transformant containing the above vector for tomato genome editing;

(3) the above target sequence;

(4) the mutant sequence of the Solyc03g053130 gene set forth in SEQ ID NO:9.

In the above material, the microorganism transformant containing the above vector for the tomato genome editing is LBA4404 containing pKSE401-sgRNA.

In the above materials, the mutant sequence of the Solyc03g053130 gene (SEQ ID NO: 9) is a sequence obtained by inserting a thymine (T) between position 1605 and position 1606 of the Solyc03g053130 gene (SEQ ID NO: 1) of a wild-type tomato and keeping the other sequences unchanged.

Still another object of the present invention is to provide a novel use of the above materials or the male sterile lines of tomato obtained by the above methods.

The present invention provides a use of the above vector or microorganism transformant or target sequence or mutant sequence for breeding a male sterile line of tomato.

The present invention also provides a use of the above vector or microorganism transformant or target sequence or mutant sequence or a male sterile line of tomato obtained by the above methods for tomato breeding.

The use of the above Solyc03g053130 protein or its encoding gene for breeding a male sterile line of tomato is also within the protection scope of the present invention.

Yet another object of the present invention is to provide a method for identifying or assisting in identifying whether a tomato to be tested is a male sterile plant.

The method for identifying or assisting in identifying whether a tomato to be tested is a male sterile plant provided by the present invention comprises the following steps:

detecting the genotype of the tomato to be tested, and determining whether the tomato to be tested is a male sterile plant according to its genotype;

if the genotype of the tomato to be tested is T/T, the tomato to be tested is a male sterile plant or a candidate male sterile plant;

if the genotype of the tomato to be tested is G/G or T/G, the tomato to be tested is a male fertile plant or a candidate male fertile plant;

the T/T genotype is a homozygote in which the base at position 1606 of each tomato Solyc03g053130 gene is T;

the G/G genotype is a homozygote in which the base at position 1606 of each tomato Solyc03g053130 gene is G;

the T/G genotype is a heterozygote of T and G at position 1606 of the tomato Solyc03g053130 gene.

In the above methods, the method for detecting the genotype of the tomato to be tested is performing PCR amplification using a set of primers to obtain an amplification product, and detecting the genotype of SNP 1606 locus in the amplification product; the SNP1606 locus is position 1606 in tomato Solyc03g053130 gene.

In the above methods, the set of primers is composed of primer 1, primer 2 and primer 3;

the primer 1 is a DNA molecule set forth in SEQ ID NO: 10;

the primer 2 is a DNA molecule set forth in SEQ ID NO: 11;

the primer 3 is a DNA molecule set forth in SEQ ID NO: 12.

In the above methods, the genotype of SNP 1606 locus in the amplification product is detected using the ArrayTape platform.

A final object of the present invention is to provide a product for identifying or assisting in identifying whether a tomato to be tested is a male sterile plant.

The product for identifying or assisting in identifying whether a tomato to be tested is a male sterile plant provided by the present invention is any one of the following (1) to (3):

(1) the above set of primers;

(2) a PCR reagent comprising the set of primers described in ,

(3) a kit comprising the set of primers described in (1) or the PCR reagent described in (2).

The use of the above methods for identifying or assisting in identifying whether a tomato to be tested is a male sterile plant or the above products for breeding a male sterile plant of tomato is also within the protection scope of the present invention.

The nucleotide sequence of the Solyc03g053130 gene of the present invention is SEQ ID NO: 1 in the sequence listing; the male sterile line of tomato refers to the tomato plant whose pollen is shrunken and aborted, and whose seeds cannot be normally harvested by self-crossing but can be harvested by pollination with the pollen of other normal tomato plants.

### DESCRIPTION OF THE DRAWINGS

FIG. 1 is a diagram showing the structure of the Solyc03g053130 gene in Example 1.

FIG. 2 is an agarose gel electrophoretogram of the T₀ generation tomato in Example 2.

FIG. 3 is a diagram showing the mutation types of the T₀ generation tomato in Example 2.

FIG. 4 is an agarose gel electrophoretogram of the Ti generation tomato in Example 2.

FIG. 5 is a sequencing peak chromatogram of the homozygous mutant in Example 2.

FIG. 6 is an image showing the fluorescein diacetate staining of pollenin Example 3.

FIG. 7 is picture showing the fruit setting results in the self-crossing and hybridization of the homozygous mutant in Example 3.

FIG. 8 is a map showing the typing of the partial F2 population by detecting the SNP 1606 using the KASP marker in Example 4. Red represents homozygous mutation T/T, blue represents homozygous mutation G/G, and green represents heterozygous mutation T/G.

### DETAILED DESCRIPTION OF THE INVENTION

The experimental methods used in the following examples are conventional methods unless otherwise specified. For example, the conditions are according to Molecular Cloning: A Laboratory Manual (Second Edition, Edited by: J. Sambrook, et al., Translated by: Huang Peitang, et al., Science Press, 2002), or recommended by the manufacturer.

The materials, reagents and the like used in the following examples are commercially available unless otherwise specified.

In the quantitative experiments in the following examples, three replicates were set and the results were averaged.

The tomato line used in the following examples is Moneymaker, and the public can obtain it from the Beijing Academy of Agricultural and Forestry Sciences or the Institute of Genetics and Developmental Biology of the Chinese Academy of Sciences.

The pKSE401 and pMD18-T vectors in the following examples were purchased from the Addgene vector library (http://www.addgene.org/); Premix Taq DNA polymerase, PrimeSTAR HS DNA polymerase and DNA ligation kit (DNA Ligation Kit Ver.2.1) were all purchased from Dalian TaKaRa Company; the restriction endonucleases were purchased from NEB; the PCR product purification kit was purchased from Omega; the shortcut plant genomic DNA extraction kit was purchased from Biomed; the primers were synthesized by Thermo Fisher Scientific; sequencing was performed by Beijing Ruiboxingke Company; the remaining reagents were analytically pure reagents.

EXAMPLE 1

### Construction of CRISPR/Cas9 Gene Editing Vector Containing Solyc03g053130 Gene-Specific sgRNA Target

1, SEQ ID NO: I is the nucleotide sequence of the Solyc03g053130 gene, and its structure is shown in FIG. 1. The sequence set forth in positions 1-1528 is the promoter sequence, the sequence set forth in positions 1529-1920 is the Exon 1 sequence, the sequence set forth in positions 2089-2374 is the Exon 2 sequence, the sequence set forth in positions 2461-2628 is the Exon 3 sequence, and the sequence set forth in positions 2737-3137 is the Exon 4 sequence. The Solyc03g053130 gene sequence set forth in SEQ ID NO: 1 was submitted to the CRISPRdirect online target analysis database (http://crispr.dbcls.jp/), the PAM sequence was set to NGG, and the species data was set to Tomato (Solanum lycopersicum) str. Heinz 1706 genome SL2.50 for CRIPSR/Cas9 target design. The reverse complementary sequence of positions 1601-1619 (Exon 1) of SEQ ID NO: 1 was finally selected as the sgRNA target sequence for editing the Solyc03g053130 gene.

The sgRNA target sequence of the Solyc03g053 30 gene is as follows: 5′-gggaaagaagaaacaagtg-3′ (SEQ ID NO: 2).

2. Primer pair Oligo-01F and Oligo-R containing the above sgRNA target sequence was synthesized, and the primer sequences are as follows:
Oligo-01F: 5′-attggggaaagaagaaacaagtg-3′ (SEQ ID NO: 3);
Oligo-R: 5′-aaaccacttgtttcttctttccc-3′ (SEQ ID NO: 4).

3. The above primer pair Oligo-01F and Oligo-R was annealed, and ligated to the binary vector pKSE401 digested with Bsa I to obtain a recombinant vector pKSE401-sgRNA. The recombinant vector pKSE401-sgRNA was transformed into E. coli DH5α, and the positive clones were selected for sequencing. Please refer to the literature “Xing, H. L., Dong, L., Wang, Z. P., Zhang, H. Y., Han, C. Y., Liu, B., Wang, X. C., and Chen, Q. J. (2014). A CRISPR/Cas9 toolkit for multiplex genome editing in plants. BMC plant biology 14:327” for specific steps.

The sequencing results showed that the recombinant vector pKSE401-sgRNA was a vector obtained by inserting the DNA molecule set forth in SEQ ID NO: 2 between the Bsa I restriction sites of pKSE401 vector and keeping the other sequences of pKSE401 vector unchanged.

4. After sequencing, the correct clones were selected and plasmid was extracted and then was transformed into Agrobacterium tumefaciens LBA4404 (Beijing Huayueyang biotechnology co. LTD, NRR01270) to obtain the bacteria liquid of Agrobacterium tumefaciens LBA4404-C 1 containing the CRISPR/Cas9 gene editing vector pKSE401-sgRNA..

Example 2. Acquisition and identification of transgenic tomato with edited Solyc03g053130 gene by CRISPR/Cas9

I. Acquisition of transgenic tomato with edited Solyc03g053130 gene by CRISPR/Cas9

1. Preparation of media related to transformation of tomato

LB liquid medium is a medium obtained by mixing tryptone, yeast extract, NaCl and water, wherein the concentration of tryptone in the LB liquid medium is 10 g/L, the concentration of yeast extract in the LB liquid medium is 5 g/L, and the concentration of NaCl in the LB liquid medium is 10 g/L.

MS liquid medium: 4.4 g of MS salt (purchased from Beijing Huayueyang biotechnology co. LTD, item No.: M519), 30 g of sucrose and water were mixed, the volume was brought to 1 L with water, and the mixture was adjusted to pH 5.8-6.0 with 1 mol/L KOH. The resulting mixture was subjected to autoclaved sterilization.

Seed growth medium (1/2 MS medium): 2.2 g of MS salt, 30 g of sucrose and water were mixed, the volume was brought to 1 L with water, and the mixture was adjusted to pH 5.8-6.0 with 1 mol/L KOH and 0.8% agar was added. The resulting mixture was subjected to autoclaved sterilization.

Pre-(co-) culture medium (D1): 4.4 g of MS, 1.0 mg of zeatin and 30 g of sucrose were dissolved in water, the volume was brought to 1 L with water, and the mixture was adjusted to pH 5.8-6.0 with 1 mol/L KOH and 0.8% agar was added. The resulting mixture was subjected to autoclaved sterilization.

Screening and differentiation medium (2Z): 4.4 g of MS salt, 2.0 mg of zeatin, 50 mg of kanamycin, 100 mg of inositol, 0.5 mg of folic acid and 20 g of sucrose were dissolved in water, the volume was brought to 1 L with water, and the mixture was adjusted to pH 5.8-6.0 with 1 mol/L KOH and 0.8% agar was added. The resulting mixture was subjected to autoclaved sterilization.

Rooting medium: 4.4 g of MS salt, 50 mg of kanamycin, 0.5 mg of folic acid, 0.5 mg of indolebutyric acid and 30 g of sucrose were dissolved in water, the volume was brought to 1 L with water, and the mixture was adjusted to pH 5.8-6.0 with 1 mol/L KOH and 0.8% agar was added. The resulting mixture was subjected to autoclaved sterilization.

2. Preparation of transgenic tomato with edited Solyc03g053130 gene by CRISPR/Cas9

(1) Preparation of Transformed Explants

The big-plump seeds of the wild-type tomato Moneymaker were selected, soaked in 40% NaCl for 20 min, rinsed with sterile water for 5 times, sown on the seed growth medium, and cultured at 25° C. with 16 h light/8 h darkness, After 8 days of germination, the cotyledons were cut into small squares under aseptic conditions with sharp scissors (the action should be fast), and the squares of the cotyledons were inoculated into the pre-culture medium and cultured at 25° C. with 16 h light/8 h darkness. After two days, it can be used for the transformation of tomato.

(2) Preparation of Infecting Solution

The LBA4404-C1 stored for use was inoculated in LB liquid medium containing kanamycin and rifampicin antibiotics, and cultured overnight at 28° C., 200 rpm. The next day, the culture was transferred to a new LB liquid medium at a ratio of 1:100, and cultured at 28° C., 200 rpm until OD₆₀₀=0.8. The bacterial solution was centrifuged at 5000 rpm for 10 min, and the supernatant was discarded to collect the bacteria. The bacteria were resuspended in MS liquid medium, diluted to OD₆₀₀=0.4, and 50 μL of 0.074 mol/L acetosyringone was added. The resulting infecting solution was stored for use.

(3) Transformation, Screening and Rooting of Explants

The squares of the cotyledons obtained in step (1) were separately immersed in the infecting solution prepared in step (2) for 10 min, and then inoculated in D1 medium (a filter paper was placed on the medium) for two days, and transferred to the screening and differentiation medium (2Z) for screening culture, subcultured every 2 weeks, and resistant buds were produced after 8 weeks of culture. When the adventitious buds elongated to 3 cm, the resistant buds were cut with a scalpel and transferred to rooting medium for rooting culture, and the rooted T₀ generation transgenic plants were transferred to soil for routine management and molecular identification. T₀ generation transgenic plants were self-crossed to obtain the seeds of T₁ generation transgenic tomato.

The conditions of the above co-culture, screening culture and rooting culture were all as follows: temperature was 25° C., 16 h light/8 h darkness.

II. Identification of T₀ generation transgenic plants and acquisition of T₁ non-transgenic homozygous mutant plants

1. The genomic DNAs of the leaves of the wild-type tomato Moneymaker and the T₀ generation transgenic plants were extracted, respectively.

2. Using the genomic DNA in step 1 as a template, PCR amplification was carried out using a primer pair consisting of CAS9-F and CAS9-R to obtain a PCR amplification product. PCR amplification conditions: 3 minutes of pre-denaturation at 94° C., 35 cycles of 30 seconds of denaturation at 94° C., 30 seconds of anneal at 55° C. and 30 seconds of extension at 72° C., and 10 minutes of extension at 72° C. in the final cycle.

CAS9-F: 5′-tcaactgagcaaagacacct-3′ (SEQ ID NO: 5);

CAS9-R: 5′-ctcgtacagcagagagtgtt-3′ (SEQ ID NO: 6).

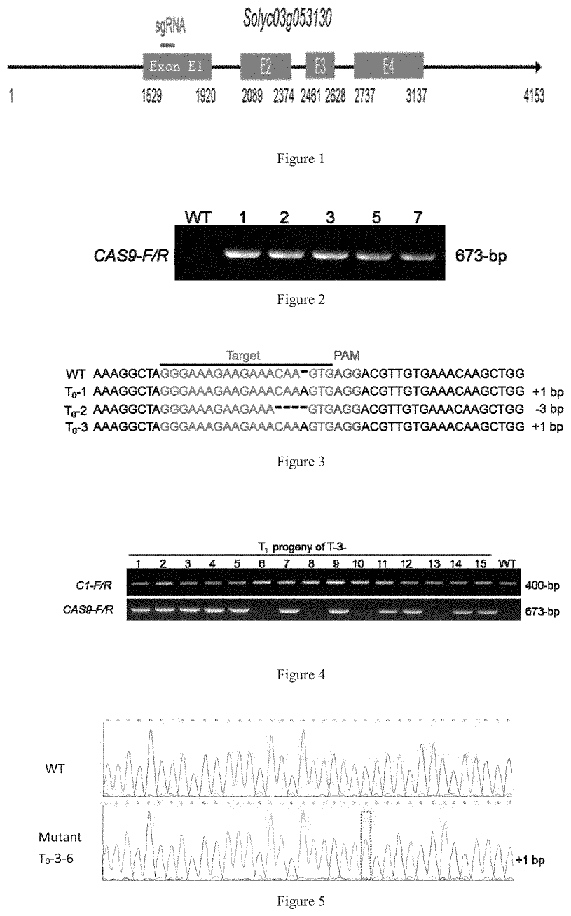

3. The PCR amplification product of step 2 was subjected to 1% agarose gel electrophoresis. The result is shown in FIG. 2. It can be seen from FIG. 2 that the PCR amplification product using the genomic DNA of the wild-type tomato as the template had no specific band in the agarose gel electrophoresis detection, while the PCR amplification products using the genomic DNAs of the T₀ generation transgenic tomato plants (1, 2, 3, 5, 7) as the templates showed a specific band of 673 bp in agarose gel electrophoresis, indicating that the T₀ generation transgenic tomato plants contain a CAS9 transgenic fragment.

4. Using the genomic DNA in step 1 as a template, PCR amplification was carried out using a primer pair consisting of C1-F and C1-R to obtain a PCR amplification product. PCR amplification conditions: 3 minutes of pre-denaturation at 94° C., 35 cycles of 30 seconds of denaturation at 94° C., 30 seconds of anneal at 55° C. and 30 seconds of extension at 72° C., and 10 minutes of extension at 72° C. in the final cycle.

C1-F: 5′-tctccgaccagttacgtgtgac-3′ (SEQ ID NO: 7);

C1-R: 5′-atgcctatcaacgatcctcacat-3′ (SEQ ID NO: 8).

5. The PCR amplification product in step 4 was inserted into the pMD18-T vector, transformed into E. coli DH5α, and 20 positive clones were selected for each PCR amplification product for sequencing. Comparing the sequencing results of the T₀ generation transgenic tomato with that of the wild-type, the result showed that there were multiple mutation types in the target segment of the T₀ generation transgenic tomato, as shown in FIG. 3. A transgenic plant No. 3 in which one base was inserted into the target segment was selected for subsequent analysis.

6. The transgenic plant No. 3 was self-crossed and the T₁ generation transgenic tomato seeds were harvested. The T₁ generation transgenic tomato was sown in a seedling tray, and the genomic DNA of each individual plant was extracted at two-euphylla one-bud stage. The individual plant containing no CAS9 transgenic fragment screened by the methods in steps 2 and 3 was used for subsequent analysis, such as the individual plant Nos.: T₀-3-6, T₀-3-8, T₀-3-10 and T₀-3-13 in FIG. 4.

7. The genomic DNA of the individual plant containing no CAS9 transgenic fragment was used as a template, and PCR amplification was carried out using a primer pair consisting of C1-F and C1-R to obtain a PCR amplification product. The PCR product was purified and sequenced, and the homozygous Solyc03g053130 mutant plant (the same mutation occurred in the two Solyc03g053130 genes of two homologous chromosomes) in which one base was inserted into the target segment, i.e., the individual plant No, T₀-3-6 in FIG. 5 (the figure shows the insertion of one base (adenine A) into the target sequence) was identified. Since the target sequence is the reverse complementary sequence of positions 1601-1619 of SEQ ID NO: 1, it results in the insertion of a thymine (T) between position 1605 and position 1606 in the corresponding Solyc03g053130 gene sequence and a frameshift mutation in the first exon, and the mutant sequence of the Solyc03g053130 gene is set forth in SEQ ID NO: 9 in the sequence listing.

The homozygous Solyc03g053130 mutant plant (individual plant No. T₀-3-6) is a plant obtained by inserting a thymine (T) between position 1605 and position 1606 of each Solyc03g053130 gene of the two homologous chromosomes of the wild-type tomato Moneymaker and keeping the other sequences of the genome of the wild-type tomato Moneymaker unchanged.

### EXAMPLE 3

### Pollen Viability Detection and Fertility Detection of Homozygous Solyc03g053130 Mutant Plant

I. Pollen viability detection of homozygous Solyc03g053130 mutant plant

1. Reagent Configuration

Fluorescein diacetate (FDA) mother liquor: 10 mg of FDA was taken and dissolved in 5 mL of acetone, dispensed into 1.5 mL centrifuge tubes, and stored at −20° C., protected from light. BK buffer S15 MOPS (pH 7.5) buffer: 5 mL of MOPS (100 mM, pH 7.5), 7.5 g of sucrose, 6.35 μL of Ca(NO₃)₂ (1 M), 4.05 μL of MgSO₄ (1 M) and 5 μL of KNO₃ (1 M) were dissolved in water, and the volume was brought to 50 mL with water. The buffer was dispensed into 1.5 mL centrifuge tubes and stored at −20° C., protected from light.

2. Fluorescein Diacetate Staining of Pollen and Observation

(1) 1 μL of FDA mother liquor was added to 1 mL of BK buffer S15 MOPS buffer and mixed, and 1 drop was taken and dropped onto a clean glass slide.

(2) A small amount of pollen was taken from the anthers of the wild-type tomato Moneymaker and homozygous Solyc03g053130 mutant plant with a tweezers, respectively, placed on the mixed droplet, covered with a cover glass, and observed under blue light (wavelength: 495 nm) by fluorescence confocal microscopy.

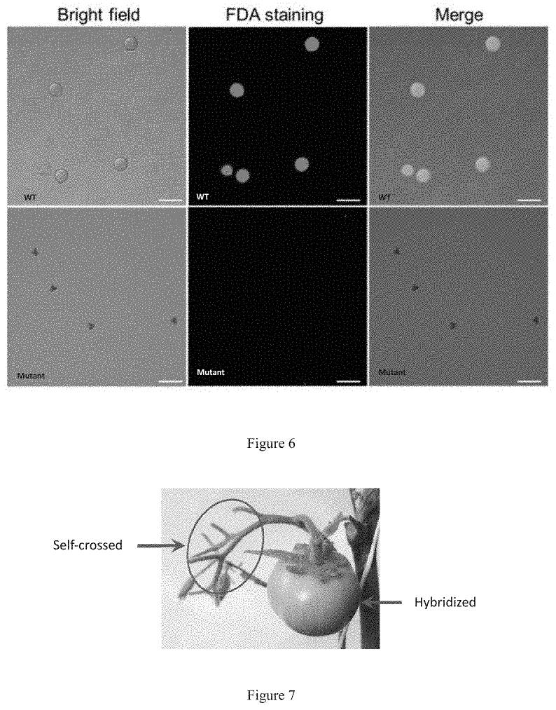

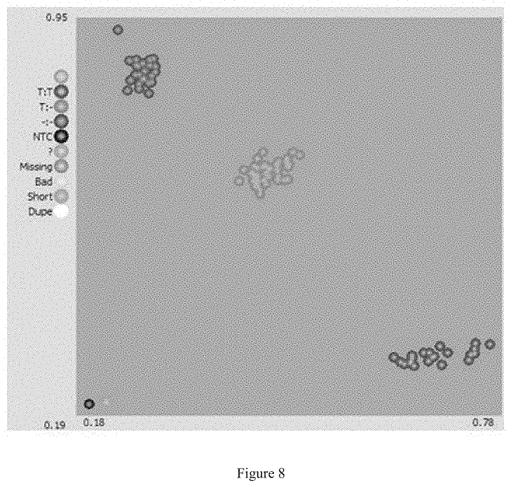

The test results are shown in FIG. 6. It can be seen from the figure that the pollen of homozygous Solyc03g053130 mutant plant (individual plant No. T₀-3-6) was smaller and more shrunken than the pollen of the wild-type tomato plant Moneymaker; under blue light, the pollen of the wild-type was green, while the pollen of homozygous Solyc03g053130 mutant plant (individual plant No. T₀-3-6) had no staining signal, indicating that the pollen of homozygous Solyc03g053130 mutant plant (individual plant No. T₀-3-6) had no viability,

II. Fertility detection of homozygous Solyc03g053130 mutant plant

Homozygous Solyc03g053130 mutant plant (individual plant No. T₀-3-6) was unable to produce seed by self-crossing. When the wild-type tomato Moneymaker used as the male parent was hybridized with the homozygous Solyc03g053130 mutant plant (individual plant No. T0-3-6) used as the female parent, viable Fl seeds could be obtained, However, when the wild-type tomato Moneymaker used as the female parent was hybridized with the homozygous Solyc03g053130 mutant plant (individual plant No. T₀-3-6) used as the male parent, the F 1 seeds could not be obtained (FIG. 7). This indicates that the homozygous mutant is male-sterile and female-fertile,

### EXAMPLE 4

### Application of a Specific Molecular Marker in Assisted Identification of Male Sterile Plants in Hybrid Progeny

A thymine (I) was inserted between the position 1605 and position 1606 of the Solyc03g053130 gene sequence in the homozygous male-sterile mutant (homozygous Solyc03g053130 mutant plant), and based on this mutation type, a Kompetitive Allele Specific PCR (KASP) molecular marker was developed based on the ArrayTape detection platform of Douglas Scientific to assist in identification of male sterile plants.

I. Method for assisting in identifying male sterile plants in hybrid progeny by using a specific molecular marker

1. Design of Primer Combination

The nucleotide at position 1606 of the Solyc03g053130 gene set forth in SEQ ID NO: 1 was named SNP1606. If both of the bases of SNP1606 locus of the tomato Solyc03g053130 gene are T, the individual is a homozygous individual and the genotype of the individual is named T/T genotype; if both of the bases of SNP1606 locus of the tomato Solyc03g053130 gene are G, the individual is a homozygous individual and the genotype of the individual is named G/G genotype; if the bases of the SNP 1606 locus of the tomato Solyc03g053130 gene are T and G, the individual is a heterozygous individual and the genotype of the individual is named T/G genotype.

The genotype of the wild-type tomato is G/G, while the genotype of the homozygous male-sterile mutant (homozygous Solyc03g053130 mutant plant) is T/T. According to the mutation site SNP1606, specific primer combination (FP1 FP2 and RP) was designed. The primer sequences are as follows:

FP1: 5′-gaaggtgaccaagttcatgctaaaggctagggaaagaagaaacaag-3′ (SEQ ID NO: 10);

FP2: 5′-gaaggtcggagtcaacggattcaaaggctagggaaagaagaaacaaa-3′ (SEQ ID NO: 11);

RP: 5′-gatccaattgataagaagccagcttgtt-3′ (SEQ ID NO: 12).

2. PCR Amplification

Using the genomic DNAs of the wild-type tomato and the homozygous male-sterile mutant (homozygous Solyc03g053130 mutant plant) as templates, respectively, and the primers designed in step 1 were used for PCR amplification. The amplification products were sequenced.

1.6 μL PCR reaction system for the ArrayTape platform detection comprises: 0.8 μL of 50 ng/μL genomic DNA, 0.03 μL of primer mix (the final concentration of the forward primers FP1 and FP2 in the system was 12 pmol·L⁻¹, the final concentration of the reverse primer RP in the system was 24 pmol·L⁻¹) and 0.8 μL of 2×KASP Mix (StdRox) from LGC.

PCR amplification procedure: 1 cycle of 10 minutes of pre-denaturation at 95° C., 40 cycles of 20 seconds of denaturation at 95° C., 60 seconds of anneal at 55° C. The above PCR amplification system was detected by the ArrayTape platform from Douglas Scientific, and the experiment was repeated twice.

3. Data Record

The data read by the built-in software of the instrument can divide different genotype data.

The genotype data of homozygous loci can be recorded as Allele1/Allele1 or Allele2/Allele2, and the genotype data of the heterozygous locus can be recorded as Allele1/Allele2. Among them, Allele1 and Allele2 represent the two allele bases at the mutation site are T and G, respectively, therefore, the genotype represented by Allele1/Allele1 is T/T, and the genotype represented by Allele2/Allele2 is G/G, the genotype represented by Allele1/Allele2 is T/G.

After verified by sequencing, the detection results using the KASP marker were consistent with the sequencing results, which proved that the detection method was credible.

Therefore, the following method can be used to assist in identifying whether the tomato to be tested in the hybrid progeny is a male sterile plant or a male fertile plant: detecting the genotype of the tomato to be tested, and determining whether the tomato to be tested is a male sterile plant or a male fertile plant according to its genotype,

if the genotype of the tomato to be tested is T/T, the tomato to be tested is a male sterile plant or a candidate male sterile plant;

if the genotype of the tomato to be tested is G/G or T/G, the tomato to be tested is a male fertile plant or a candidate male fertile plant.

II. Verification of the method for assisting in identifying male sterile plants in hybrid progeny by using the specific molecular marker

The KASP marker was used to assist in identification of 201 male sterile plants in F2 population. Specific steps were as follows:

1. The wild-type tomato Moneymaker was used as the male parent, and the homozygous Solyc03g053130 mutant plant (individual plant No. T₀-3-6) was used as the female parent to carry out the hybridization, and the F1 generation seeds were harvested.

2. The F1 generation seeds were cultivated to be plants, i.e., F1 generation plants.

3. The F1 generation plants were self-crossed and F2 generation seeds were harvested.

4. The F2 generation seeds were cultivated to be plants, i.e., F2 generation plants.

5. The genotypes of SNP1606 loci of 201 plants of F2 generation were detected by using the KASP marker according to the method in step I. The male fertility of 201 plants of F2 generation was determined by using the fluorescein diacetate staining method (refer to Example 3 for specific steps) in combination with the field observation (determining whether they could be self-crossed to produce seeds).

The detection results of the KASP marker showed that the genotype of SNP1606 locus of 51 plants of F2 generation were T/T, and these plants were all male sterile plants, indicating that the KASP marker can accurately identify male sterile plants in the hybrid population.

### INDUSTRIAL APPLICATION

The present invention uses the CRISPR/Cas9 genome editing technology to rapidly create a male sterile line of tomato, and develops a molecular marker which can assist in identifying whether the tomato plant to be tested is a male sterile line. The method for creating a male sterile line of tomato and the method for detecting a male sterile line can be applied to other tomato lines. Compared with the traditional backcrossing trans-breeding, this method can greatly shorten the trans-breeding time of male infertility. The primal parent can be transferred from a male fertile line to a male sterile line within 1-2 years, and there is no adverse effect such as linkage drag. It has great application prospect and economic value in breeding.

What is claimed is:

1. A method for breeding a male sterile line of tomato, comprising the following steps: using a CRISPR/Cas9 system to edit a fertility gene in the genome of a recipient tomato, thereby losing the function of the fertility gene and obtaining the male sterile line of tomato.

2. The method according to claim 1, wherein the fertility gene is a gene encoding a Solyc03g053130 protein;
the Solyc03g053130 protein is the following 1) or 2):
1) a protein with the amino acid sequence set forth in SEQ ID NO: 13;
2) a protein derived from 1), which is obtained by substitution, deletion and/or addition of one or more amino acid residues in the amino acid sequence set forth in SEQ ID NO: 13.

3. The method according to claim 1, wherein the CRISPR/Cas9 system comprises a sgRNA;
the target sequence of the sgRNA is the DNA molecule set forth in SEQ ID NO: 2.

4. The method according to claim 1, wherein the editing method is introducing a vector for tomato genome editing into the recipient tomato.

5. The method according to claim 4, wherein the vector for tomato genome editing is a vector obtained by inserting the DNA molecule set forth in SEQ ID NO: 2 between the Bsa I restriction sites of pKSE401 vector and keeping the other sequences of pKSE401 vector unchanged.

6. The method according to claim 1, wherein the method further comprises the step of screening a homozygous Solyc03g053130 mutant.

7-10. (canceled)

11. A method for identifying or assisting in identifying whether a tomato to be tested is a male sterile plant, comprising the following steps: detecting the genotype of the tomato to be tested, and determining whether the tomato to be tested is a male sterile plant according to its genotype;
if the genotype of the tomato to be tested is T/T, the tomato to be tested is a male sterile plant or a candidate male sterile plant;
if the genotype of the tomato to be tested is G/G or T/G, the tomato to be tested is a male fertile plant or a candidate male fertile plant;
the T/T genotype is a homozygote in which the base at position 1606 of each tomato Solyc03g053130 gene is T;
the GIG genotype is a homozygote in which the base at position 1606 of each tomato Solyc03g053130 gene is G;
the T/G genotype is a heterozygote of T and G at position 1606 of the tomato Solyc03g053130 gene.

12. The method according to claim 11, wherein the method for detecting the genotype of the tomato to be tested is performing PCR amplification using a set of primers to obtain an amplification product, and detecting the genotype of SNP 1606 locus in the amplification product;
the SNP1606 locus is position 1606 in tomato Solyc03g053130 gene.

13. The method according to claim 12, wherein the set of primers is composed of primer 1, primer 2 and primer 3;
the primer 1 is a DNA molecule set forth in SEQ ID NO: 10;
the primer 2 is a DNA molecule set forth in SEQ ID NO: 11;
the primer 3 is a DNA molecule set forth in SEQ ID NO: 12.

14. The method according to claim 12, wherein the genotype of SNP 1606 locus in the amplification product is detected using the ArrayTape platform.

15. A product for identifying or assisting in identifying whether a tomato to be tested is a male sterile plant, which is any one of the following (1) to (3):
(1) the set of primers in claim 13;
(2) a PCR reagent comprising the set of primers described in (1);
(3) a kit comprising the set of primers described in (1) or the PCR reagent described in (2).

16. (canceled)
